(19) United States
(12) Patent Application Publication
(10) Pub. No.: US 2002/0119192 A1
(43) Pub. Date: Aug. 29, 2002

(54) Controlled release formulations for oral administration

(76) Inventors: Narayanan Badri Vishwanathan, Chennai (IN); Rajeev S. Raghuvanshi, New Delhi (IN); Gour Mukherji, Haryana (IN); Ashok Rampal, Punjab (IN)

Correspondence Address: JAYADEEP R. DESHMUKH, RANBAXY PHARMACEUTICALS INC., SUITE 2100, 600 COLLEGE ROAD EAST, PRINCETON, NJ, 08540, US

(21) Appl. No.: 09/957,799

(22) Filed: Sep. 21, 2001

(30) Foreign Application Priority Data
Sep. 22, 2000 (IN) 856/DEL/2000
Aug. 24, 2001 (IN) 881/DEL/2001

(51) Int. Cl. A61K 9/62; A61K 9/58

(52) U.S. Cl. 424/461; 424/462

(57) ABSTRACT

A pharmaceutical composition in the form of an oral controlled release solid dosage form comprising an effective amount of drug, or its pharmaceutically acceptable salts. It also relates to a pharmaceutically composition that is suitable for once-a-day dosing regimen.

### FIELD OF THE INVENTION

The invention relates to a pharmaceutical composition in the form of an oral controlled release solid dosage form comprising an effective amount of drug or its pharmaceutically acceptable salts.

### BACKGROUND OF THE INVENTION

The most commonly employed route of administration of drugs/medications is oral ingestion. However, an oral formulation is subjected to highly variable conditions during its transit through gastro-intestinal tract. Also, the salient aspect of drug delivery by known conventional immediate release dosage forms, is the fluctuation between high and low concentrations within each dosing interval. The traditional pharmaceutical forms generally release the drug in a period of few minutes to 2 hours after administration, making frequent ingestion of multiple doses per day, a necessity. Such an administration results in wide variations in serum concentration throughout the course of treatment. The approach to control the duration of drug release and to maintain the drug at a predictable, specified concentration in vivo, offers many advantages over other forms of medication.

The convenience of using a drug delivery system which releases drug over a prolonged period of time as opposed to the immediate release formulations, has long been recognized in the practice of medicine. The therapeutic advantage which inures to the benefit of the patient and the clinician is controlled and optimum blood levels during the period of drug release from the delivery system, thereby, also reducing symptomatic side effects. Further, another advantage of longer acting drug preparations would be both increased convenience and improved patient compliance resulting from the avoidance of missed doses through patient forgetfulness. Oral controlled release delivery systems should ideally be adaptable so that release rates and profiles can be matched to physiological and chronotherapeutic specifications.

To design an optimum oral controlled release system, it is necessary to take into account the physicochemical and physiological environment of the gastrointestinal tract. The gastric residence time is subject to very significant interindividual variations and is inter alia dependent on the nutritional habits of the individual. Meals of high calorific value, especially fats, have an inhibitory effect on gastric emptying. However, upon oral administration, the drug formulation usually traverses the stomach in about 1-2 hours. This relatively short gastric residence time necessitates frequent oral administration for drugs having an “absorption window” in the upper gastrointestinal tract. Conventional approaches to controlled release formulation known in the art are not applicable to such a class of drugs. Slow release formulations of such drugs may only be effective for about 4-5 hours whereafter the formulation passes into the colon and the drug absorption reaches the minimum. Retention of drug formulations in the proximal region of the gastrointestinal tract, and for controlled release of such drugs in such region has been a long sought objective. One example of such a drug whose bioavailability is highly dependent on the local physiology of the gastrointestinal tract, is ofloxacin. Ofloxacin is readily soluble in the acidic environment of the stomach. In 0.1N HCl media, it is classified as “soluble” having a solubility of 5.8% w/v. In buffer of pH 4.5, ofloxacin is “slightly soluble” with solubility of only about 0.9% w/v. In media of still higher pH (about 6.8) the solubility falls even more drastically. This pH related solubility adversely affects drug dissolution and hence absorption in the gastrointestinal tract. Due to greater solubility of ofloxacin in lower pH media, better drug bioavailability can be ensured by extended residence of the dosage form in the stomach. In the intestine, where pH is normally above 4.0, the release of the drug may be adversely affected owing to lower solubility which may further influence the rate and extent of drug absorption, thus affecting both peak plasma concentration (Cmax) and bioavailability (Area Under the Curve, AUC). It is readily apparent that controlled release formulations which are retained in the stomach and which slowly release medicament over an extended period of time would be eminently suited for this class of drugs in general and ofloxacin in particular. Such a controlled release dosage form is provided by the present invention.

The literature discloses various approaches for therapeutic dosage forms which are designed to deliver the drug in the upper regions of the gastrointestinal tract and possess controlled release characteristics.

One such approach mentioned in prior art makes use of a therapeutic system which swells highly in the stomach and consequently cannot pass through the pylorus. U.S. Pat. No. 5,780,057, for example, describes a pharmaceutical tablet having a multilayer structure wherein at least one layer swells remarkably in the presence of biological aqueous fluids resulting in an increase by at least 50% of the total volume of the tablet and thereby exhibits a high residence time in the stomach and/or in the upper portion of the gastrointestinal tract. Said layer, being a granular mixture of biocompatible hydrophilic polymers and highly swellable (super disintegrating) polymers, acts as a barrier and modulates the slow release of the active ingredient from the pharmaceutical dosage form. The expanded dosage forms may block the pyloric sphincter or may cause unfavourable conditions following multiple to dosing resulting from retention of swollen dosage units in the stomach.

U.S. Pat. No. 5,651,985 discloses a composition comprising 30-90%, by weight of the composition, homogenous mixture of polymers containing lactam groups and polymers containing carboxyl groups as gel forming agents which markedly swells to form a gel of high mechanical and dimensional stability in the aqueous environment of the stomach. The swelling polymers required for such dosage forms are in very high concentration which makes such delivery systems very large and inconvenient for oral administration of high dosage medicaments, such as ofloxacin with a daily dose requirement of 400-800 mg.

Other techniques which have been described in the prior art for increasing the gastric residence time include buoyant systems which float in the gastric fluids solely as a result of the low specific gravity of its matrix formulation. U.S. Pat. No. 4,126,672 discloses formulations comprising finely particulate, homogeneous mixture of chlordiazepoxide and diazepam in combination with a hydrocolloid or mixtures of hydrocolloids so as to have bulk density of less than one and be hydrodynamically balanced when in contact with gastric fluid. U.S. Pat. No. 4,167,558 relates to formulations comprising a homogenous mixture of acetylsalicylic acid with a hydrocolloid which are hydrodynamically balanced so that, in contact with gastric fluid, they possess a bulk density of less than one and therefore are buoyant in gastric fluid and thus are retained in the stomach during the time when substantially all of the medicaments are released therefrom. The composition exemplified in these prior arts contain very high amounts of polymers (hydrocolloids) which upon contact with gastric fluids swell to form a soft gelatinous mass of bulk density less than one which thereafter slowly dissolves to release the medicament. The release of medicament is also said to take place by leaching action at or near the surface. However, it is well recognized that the application of such a system to obtain the desired rate of release of the drug wherein it is regulated by the erosion of the polymer, is difficult to maintain. Further, as the concentration of polymers required for such a system is very high, such a system is not suitable for high dose medicaments. Furthermore, the specific gravity of digestive fluids, especially that of gastric juices is from 1.004 to 1.101 and it is well ascertainable by those skilled in formulation development that it may be difficult to maintain low specific gravity for the sustained release composition as described in these prior arts, for a prolonged period. Also, as stated in their specification, for the successful practice of their invention, the hydrocolloids utilized must hydrate in acidic pH which limits the selection of polymers for the formulation scientist.

Still other techniques are directed towards use of multiparticulate systems to increase the gastric residence time as described in U.S. Pat. No. 5,007,790. This patent unveils a sustained release oral drug dosage form comprising a plurality of solid particles of a solid-state drug dispersed within a hydrophilic, water swellable polymer that swells on imbibition of gastric fluid to increase the particle size to a level that promotes retention in the stomach over said time period, permitting dissolution of the dispersed drug and release of the resulting solution through a leaching action. The swellable polymer is said to maintain its physical integrity for at least a substantial portion of the time period during which the drug is released into the stomach and thereafter, rapidly dissolves. It is well recognized by those skilled in the art that it may be difficult to obtain the desired rate of release for a drug that has a high water solubility from multiparticulate systems as described in this patent, in which the drug first undergoes dissolution followed by release of the resulting solution by leaching action.

As mentioned above, several pharmaceutical compositions are described in the reference literature which relates to controlled release drug delivery systems. However, for the above stated reasons and because the prior art discloses either complicated devices and systems which are difficult to manufacture on the industrial scale or the components used therein are not so user friendly, the oral controlled drug delivery systems heretofore described, are not completely satisfactory.

Our U.S. Pat. No. 6,261,601 describes a pharmaceutical composition in the form of tablets or capsules which provides a combination of spatial and temporal control of drug delivery when ingested by a patient. The pharmaceutical composition constitutes an oral controlled drug delivery system, comprising a drug, a gas generating component, a swelling agent, a viscolyzing agent and optionally a gel forming polymer. The viscolyzing agent and the gel forming polymer form a hydrated gel matrix which entraps the gas, causing the tablet or capsule to retain in the stomach or upper part of the small intestine (spatial control) and also creates a tortuous diffusion path for the drug, resulting in sustained release of the drug (temporal control).

### BRIEF DESCRIPTION OF THE DRAWINGS

FIG. 1 shows the results of a crossover comparative bioavailability study between the ofloxacin OD 800 mg tablets of the present and immediate release Floxin™ tablets.

FIG. 2 shows the results of a crossover comparative bioavailability study between the ofloxacin OD 400 mg tablets of the present and immediate release Floxin™ tablets.

### SUMMARY OF THE INVENTION

It is an object of the present invention to provide an oral controlled drug delivery system of drug which:

a. comprises carboxyvinyl polymer that gelatinizes in the alkaline environment and regulates the release of drug;

b. comprises hydrophilic polymers t hat swell upon imbibition of water and further provides for controlled release of drug

c. delivers the drug at a controlled rate and exhibits reproducibility of release rate into aqueous media at the absorptive regions of the gastrointestinal tract, and

d. provides greater efficacy at comparable daily dosages of conventional immediate release formulations.

It is also an object of the present invention to provide an oral controlled release solid dosage form that maintains its physical integrity and dimensional stability when in contact with gastric fluids and achieves the optimal rate of release of drug. It is a further object of the present invention that a high dose medicament may be incorporated in the therapeutic system without the loss of any of its desirable attributes. The dosage form may preferably possess floating characteristics resulting in extended residency in gastric fluids.

The above-mentioned objects are achieved by virtue of the present invention, which relates to a pharmaceutical dosage form that selectively releases the drug in a controlled manner at the gastric levels and upper parts of the intestine over a prolonged period of time.

More particularly, the present invention describes a pharmaceutical composition for oral administration in humans for the controlled release of a therapeutic agent comprising an effective amount of drug in combination with a polymeric matrix characterized in that at least one such polymer is carboxyvinyl polymer and which constitutes at least 30% by weight of the total polymeric content, an alkaline compound and optionally, other pharmaceutically acceptable auxiliary components.

It has also been discovered that cellulose ethers, preferably, hydroxypropyl methylcellulose, when added to the pharmaceutical compositions extends the in-vitro drug release profile to about 10 hours which forms another aspect of the present invention. Further, compositions including cellulose ethers exhibit a drug release profile that is better controlled and sustained.

Hydroxypropyl methylcellulose being hydrophilic in nature hydrates to form a gel layer on exposure to aqueous fluids. The effective release of the medicament is regulated by the slow erosion of this polymer. Carboxyvinyl polymer and cellulose ethers in conjunction with additional polymers recognized in the art of pharmaceutical compounding for release retarding properties together form the controlled release matrix. The drug is entrapped within this polymeric matrix. The rate of release of drug from such a system is primarily dependent on rate of water imbibition, resultant rate of swelling of matrix, drug dissolution and diffusion from the matrix.

In a particular embodiment, the present invention provides a pharmaceutical composition for oral administration in humans for the controlled release of ofloxacin which releases more than 40% of ofloxacin in less than 4 hours and releases more than 60% of ofloxacin in less than 8 hours. It further provides a pharmaceutical composition for oral administration in humans for the once-a-day delivery system of ofloxacin which releases more than 40% of ofloxacin in less than 4 hours, releases more than 60% of ofloxacin in less than 8 hours; and substantially all ofloxacin is released within about 8-10 hours.

The present invention also comprehends a therapeutic system either in the form of beads, pellets, granules, tablets and capsules incorporating drug in a polymeric matrix and optional pharmaceutical adjuvants such as swelling agents, diluents and binders. Also, the pharmaceutical dosage form may be optionally coated with a rapidly dissolving water soluble polymer film coat.

A preferred oral once-a-day delivery system of the present invention delivers minimum therapeutic serum levels for about 20 hours and therefore may be administered as a once-a-day dosage regimen. Further, the oral controlled release solid dosage form of the present invention provides greater efficacy than provided by comparable daily dosages of conventional immediate release formulations.

The various components of the present invention are described in more detail below.

According to the present invention, the delivery system provides controlled release of at least one therapeutic agent or drug. The drug may be pharmacologically active itself or may be converted into the active form by biotransformation in the body. The drug can be any drug for which therapy would be improved as a result of controlled drug delivery and extended gastric retention.

The medicament or combination of medicaments which are amenable to controlled release therapy utilizing the novel formulations of the present invention include any of those suitable for oral administration and which are readily soluble in the acidic environment of the stomach. The present invention is not to be construed as being limited to any particular medicament or class of medicaments.

The once-a-day formulations of the subject invention are particularly amenable to the administration of medicaments which are predominantly absorbed through the upper portion of the gastro intestinal tract, drugs having pH dependent solubility, i.e., more soluble in the gastric pH as compared to the intestinal pH, drugs having stomach as a site of action which includes H-2 receptor antagonists, antacids, antimuscarinic agents, proton pump inhibitors, drugs active against H. pylori, cytoprotective agents, and the like.

Broadly, the drug of the present invention is selected from the therapeutic category of antiulcer, analgesic, antihypertensive, antibiotic, antipsychotic, anticancer, antimuscarinic, diuretic, antimigraine, antiviral, anti-inflammatory, sedatives, antidiabetic, antidepressant, antihistaminic, antiparasitic, antiepileptic, lipid lowering drugs, and mixtures thereof.

Illustrative examples of drugs that are absorbed predominantly from the upper parts of gastrointestinal tract include ciprofloxacin, ofloxacin, cyclosporin, furose-mide, metoprolol, oxprenolol, baclofen, allopurinol, sumatriptan, benazepril, enalapril, quinapril, moexipril, indolapril, olindapril, retinapril, spirapril, clilaze-prilat, lisinopril, imidapril, benazeprilat, cilazapril, captopril, delapril, fosinopril, libenzapril, pentopril, perindopril, altiopril, quinaprilat, ramipril, spiraprilat, zofenopril, and the like; all of which are suitable for use in the present invention.

Drugs having the stomach as site of action include H-2 receptor antagonists such as ranitidine, famotidine, nizatidine, bifentidine, erbrotidine, nifentidine, roxatidine and cimetidine, and the like; proton pump inhibitors like omeprazole, lansoprazole, pentoprazole, and the like; antacids like magnesium carbonate, aluminium hydroxide, magnesium oxide and simethicone, and the like; cytoprotectives such as sucralphate, carbenoxolone sodium and misoprostol, and the like; antimuscarinic agents like pirenzepine, telenzepine and propanthelene bromide, and the like; drugs active against H. Pylori like bismuth salts such as bismuth subsalicylate, tripotassium dicitratobismuthate, ranitidine bismuth citrate, and the like; antibiotics for example clarithromycin, amoxycillin, and the like; all of which are suitable for use in the present invention.

Other medicaments that are suitable for this invention are drugs having solubility in acidic pH or ones having specific absorption sites in the upper part of the gastro-intestinal tract and those that are subjected to gastro-intestinal first pass metabolism (as in some reports stomach absorption is known to bypass gastrointestinal first pass metabolism) include antihypertensive agents like verapamil, nifedipine, propranolol, nimodipine, nicardipine, amlodipine, prazosin, ketanserin, guanabenz acetate, hydralazide, carvedilol, methyidopa, levodopa, carbidopa; antivirals like acyclovir, inosine, pranobex, zidovudine (AZT), tribavirin, vidarabine; lipid lowering agents like simvastatin, pravastatin, atorvastatin and lovastatin; antipsychotic agents like selegiline; sedatives like midazolam; all of which are suitable for use in the present invention.

The drug itself or its pharmacologically active salt or ester can be used in the present invention. Moreover, combination of drugs that are typically administered together may be included as the drug component.

In a particularly preferred embodiment of the present invention the delivery system contains ofloxacin as the drug.

The amount of the drug is that which is typically administered for a given period of time. This includes a pharmaceutically effective amount of the drug which is an amount high enough to significantly positively modify the condition to be treated, but low enough to avoid serious side effects (at a reasonable benefit/risk ratio), within the scope of sound medical judgement. Preferably, the drug may be present in an amount from about 30% to about 90% by weight of the total weight of the pharmaceutical composition.

According to the present invention, the polymeric matrix comprises carboxyvinyl polymer in conjunction with other hydrophilic polymers which together regulate the release of drug. The polymers which are amenable to controlled release therapy utilizing the novel therapeutic delivery system of the present invention include any of those suitable for oral administration. The hydrophilic polymer forming the matrix in accordance with this invention is any such polymer that is non-toxic, swells upon imbibition of water and provides for controlled release of the drug. The hydrophilicity of these polymers causes the drug containing matrix to swell upon ingress of water. The hydrophilic water-soluble polymers may be used individually or in combination. Examples of polymers suitable for this invention include the polymers well known in the pharmaceutical art for their release retarding properties and may be selected from the group comprising acrylic polymers such as available as Eudragit RS 30D, Eudragit RL 30D, Eudragit NE 30D, Eudragit RSPO; natural gums as xanthan gum, karaya gum, locust bean gum, guar gum, gelan gum, gum arabic, tragacanth, carrageenan, pectin, agar, alginic acid, sodium alginate and the like.

The amount of polymer relative to the drug may vary depending on the release rate desired, nature of the polymers, their physicochemical characteristics, and other auxiliary components that may be present as an integral part of the formulation. Accordingly, the carboxyvinyl polymer forms at least 30% by weight of the total polymeric content of said composition. However, the polymers together may be present in an amount from about 2% to about 25% by weight, and preferably from about 5% to about 15% by weight of the total weight of the pharmaceutical composition.

According to the present invention the polymeric matrix comprises carboxyvinyl polymer and additionally, cellulose ethers in conjunction with other hydrophilic polymers which together regulate the release of drug suitable for once-a-day dosage regimen. Cellulose ethers which may be selected from the group comprising hydroxypropyl methylcelluloses of different grades, hydroxypropyl celluloses of different grades, hydroxyethyl cellulose, methylcellulose, hydroxypropyl ethylcellulose, hydroxyethyl methylcellulose, carboxymethyl cellulose, sodium carboxymethyl cellulose, hydroxycellulose, and the like.

According to the present invention, the controlled release dosage form comprises an alkaline compound that aids in gelatinization of the carboxyvinyl polymer. Accordingly, any well known and pharmacologically safe inorganic or organic basic compounds may be used. Examples of inorganic basic salts that may be used in the present invention include ammonium hydroxide, alkali metal salts, alkaline earth metal salts such as magnesium oxide, magnesium hydroxide, calcium hydroxide, sodium hydroxide, potassium hydroxide, lithium hydroxide, aluminium hydroxide, salts of aluminium, calcium, sodium or potassium carbonate, bicarbonate, sulfites, phosphate or citrate, composite aluminium-magnesium compounds and the like. The examples of organic basic salts that may be used in the present invention include alkanolamines such as methanolamine, ethanolamine, propanolamine, butanolamine, dimethanolamine, diethanolamine, dipropanolamine, dibutanolamine, diisopropanolamine, trimethanolamine, triethanolamine, tripropanolamine, tributanolamine, aminomethylpropanol, N-methyl glucamine, tetrahydroxypropyl ethylene diamine, and the like; alkylamines such as methylamine, ethylamine, propylamine, butylamine, diethylamine, dipropylamine, isopropylamine, and the like; organic pH buffering substances such as trihydroxymethylaminomethane, and the like.

In a preferred embodiment of the present invention, the controlled release dosage form contains gas generating agents such as salts of carbonates, bicarbonates and sulfites as the alkaline reacting compound. These agents upon contact with the acidic fluids evolve gas that becomes entrapped within the hydrated matrix and thereby helps in increasing the buoyancy of the dosage form in the gastric fluids. This extends its residency in the stomach and thus prolongs its release in the stomach and upper parts of the small intestine. That is, the system is not transported past the areas of higher solubility for drug prior to releasing all or substantially all of the drug and maximum bioavailability is attained therefrom. Furthermore, the presence of entrapped gas and its expanding pressure affects the influx of the fluids through the pores of the matrix and thus exerts both a hydrodynamic and release controlling effect.

The gas generating agents may be used alone or in combination with an acid source as a couple. The gas generating agent interacts with an acid source triggered by contact with water or simply with gastric fluid to generate carbon dioxide or sulfur dioxide that gets entrapped within the polymeric matrix and thereby extends residency of delivery system in the stomach.

Accordingly, the dosage form may contain an acid source selected from the group comprising edible organic acid, a salt of an edible organic acid or mixtures thereof. The organic acid salts that may be used as the acid source in the present invention include, for example, a monoalkali salt of an organic acid having more than one carboxylic acid functional group, a bialkali metal salt of an organic acid having more than two carboxylic acid functional groups and the like. Examples of organic acids that may be used as the acid source in the present invention include, citric acid or its salts such as sodium citrate or calcium citrate, malic acid, tartaric acid, succinic acid, fumaric acid, maleic acid or their salts, ascorbic acid or its salts such as sodium or calcium ascorbate, glycine, sarcosine, alanine, taurine, glutamic acid and the like.

The alkaline compound may be present in amounts from about 5% to about 50%, preferably from about 7% to about 35% and more preferably from about 10% to about 30% by weight of the total weight of the pharmaceutical composition.

Optionally, there may also be incorporated into the delivery system of the present invention, other conventional pharmaceutically acceptable auxiliary components known in the art of formulation development such as swelling agent, diluent and binder. It is to be borne in mind, however, that the conventional pharmaceutical auxiliary additives which might adversely affect the rate of release of the drug are not suitable for use therein.

The dosage form in accordance with the present invention may contain a swelling agent from the class of compounds commonly known as superdisintegrants which absorb large amounts of fluid and causes the hydrated gel matrix to swell significantly thereby assisting in regulating the release profile of ofloxacin over a period of time. Examples of swelling agents that may be used in the present invention include cross-linked polyvinylpyrrolidone, cross-linked carboxymethyl cellulose sodium, sodium starch glycolate, and the like. The swelling agent may be present in an amount from about 5% to about 30%, preferably from about 7% to about 25% and more preferably from about 10% to about 20% by weight of the total weight of the composition.

The dosage form may contain one or more of a water soluble and/or water dispersible diluent. Examples of water soluble diluents that may be used in the present invention include, but are not limited to lactose, calcium sulphate, mannitol, dextrates, dextrin, dextrose, sucrose and the like. Water dispersible diluents which refer to water insoluble pharmaceutical excipients that disperse readily in water include, but are not limited to, cellulose based excipients such as microcrystalline cellulose, powdered cellulose, starches such as corn starch, pregelatinised starch, clays or clay minerals such as kaolin, bentonite, attapulgite and the like.

According to the present invention the dosage form may also include a binder to provide cohesiveness to the powder mass. The binders commonly known to the pharmaceutical art may be used in the present invention. Examples of the binders are pregelatinised starch, polyvinylpyrrolidone, hydroxypropyl methylcellulose, sodium carboxymethyl cellulose, starch paste, gelatin, xanthan gum, acacia, guar gum, and the like.

The pharmaceutical dosage form may also contain other conventional pharmaceutical excipients, recognized in the art of pharmaceutical compounding such as pharmaceutical grade magnesium stearate or stearic acid and the like as a glidant, talc and the like as an anti-adherent and silicon dioxide or hydrogenated vegetable oil and the like as a lubricant which form the integral part of the delivery system.

According to the present invention, the dosage form may be prepared either in the form of pellets, beads, granules, tablets or capsules.

In those embodiments of the present invention wherein the pharmaceutical composition is in the form of capsule dosage form, the capsule shell may be of a hard gelatin or a soft gelatin type. Furthermore, the capsules made of starch or hydroxypropyl methylcellulose may also be used.

The dosage form in accordance with the present invention may be optionally coated with a rapidly dissolving water soluble film coat. Examples of water soluble polymers include hydroxypropyl methylcellulose, hydroxypropyl cellulose, and the like. The solid unit dosage form in accordance with the present invention may be coated to a weight build-up of about 1% to about 10% by weight, preferably from about 1% to about 4% by weight, of the total weight of the composition.

According to the present invention, the dosage form is prepared by blending the drug with carboxyvinyl polymer, cellulose ether polymer, hydrophilic polymers, alkaline compound and the optionally added auxiliary components including lubricants. The blend is directly compressed into tablets or may be filled into capsules.

Alternatively, the dosage form is prepared by blending the aforementioned ingredients with only a portion of the lubricants. The blend is roll compacted and then sized to obtain granules. The granules may be filled into capsules or compressed into tablets.

Alternatively still, the dosage form is formulated by preparing placebo granules of alkaline compound with a solution of cellulose ether polymer. The granules are blended with the drug, carboxyvinyl polymer, hydrophilic polymer and optionally added auxiliary components including lubricants. The blend is either directly compressed into tablets or may be filled into capsules. Alternatively, the blend is roll compacted and then sized to obtain granules which may be filled into granules or compressed into tablets.

In those embodiments of the present invention wherein the foregoing composition is in the form of spherical pellets or beads, the art of producing such dosage forms by extrusion and spheronisation techniques or techniques based on high shear granulation or fluidized bed techniques may be used. Single unit pellets can be produced on industrial scale using lozenge and troches cutting machines.

Ofloxacin is a typical example of drug having an absorption window in the proximal region of the gastrointestinal tract. It is therefore selected as a representative example for illustrating the formulations of the present invention.

### DETAILED DESCRIPTION OF THE INVENTION

The present invention is illustrated below by reference to the following examples which set forth particularly preferred embodiments. However, it should be noted that these embodiments are illustrative and are not to be construed as limiting the invention in any way.

### EXAMPLE 1

This example illustrates the present invention when the active ingredient is ofloxacin. It clearly depicts the effect of alkaline microenvironment on gelation of carboxyvinyl polymer that prolongs the release. The pharmaceutical composition is given in Table 1.

TABLE 1

| INGREDIENTS | % W/W |
|---|---|
| Ofloxacin | 66.56 |
| Sodium alginate | 0.66 |
| Xanthan gum | 1.99 |
| Carboxyvinyl polymer (Carbopol 971P) | 3.73 |
| Cross-linked polyvinylpyrrolidone | 12.42 |
| Sodium bicarbonate | 13.25 |
| Colloidal silicon dioxide (Aerosil 200) | 0.25 |
| Magnesium stearate | 1.16 |

Ofloxacin, sodium alginate, xanthan gum, carboxyvinyl polymer, cross-linked polyvinylpyrrolidone, sodium bicarbonate, colloidal silicon dioxide and a part of magnesium stearate were blended together and sifted through a sieve 355 μm mesh (British Standard Sieve (BSS) No.44). The blend was compacted on a roll-compactor and the compacts sized through 850 μm mesh (British Standard Sieve (BSS) No. 18) to obtain granules. The sized granules were blended with the remaining lubricant prior to compression into tablets.

The tablets were tested for drug release in 0.1N hydrochloric acid and pH 6.8 phosphate buffer media. The USP apparatus 2 with paddle speed at 60 rpm was used for the study. The paddles were fixed at 4.5 cm away from the base of the vessel and cylindrical baskets of 1680 μm mesh (British Standard Sieve (BSS) No.10), capped at the open end, were used as sinkers. The samples of the media were periodically withdrawn and spectrophotometrically analyzed for ofloxacin content at 327 nm. The dissolution results given in Table 2 and Table 3, shows the profiles of the formulation under discussion (Table 1) as per the present innovation, and a control formulation prepared identically except having no sodium bicarbonate.

TABLE 2

| | PERCENT OFLOXACIN RELEASED | |
|---|---|---|
| | IN 0.1N HCI MEDIA | |
| TIME (Hrs.) | REFERENCE | CONTROL |
| 1 | 27.3 | 57.9 |
| 2 | 34.1 | 80.5 |
| 4 | 46.7 | 95.7 |
| 6 | 59.6 | — |
| 8 | 78.9 | — |
| 10 | 89.7 | — |

TABLE 3

| | PERCENT OFLOXACIN RELEASED | |
|---|---|---|
| | IN pH 6.8 BUFFER | |
| TIME (Hrs.) | REFERENCE | CONTROL |
| 1 | 16.5 | 15.6 |
| 2 | 19.1 | 43.6 |
| 4 | 25.1 | 72.6 |
| 6 | 32.1 | 88.9 |

### EXAMPLE 2

This example illustrates the controlled release tablets of ofloxacin wherein sodium bicarbonate is granulated separately. The pharmaceutical composition is given in Table 4.

TABLE 4

| INGREDIENTS | % W/W |
|---|---|
| Ofloxacin | 64.09 |
| Sodium alginate | 0.64 |
| Xanthan gum | 1.93 |
| Carboxyvinyl polymer (Carbopol 971P) | 3.21 |
| Cross-linked polyvinylpyrrolidone | 12.30 |
| Polyvinylpyrrolidone | 1.93 |
| Sodium bicarbonate | 12.85 |
| Talc | 0.64 |
| Magnesium stearate | 2.41 |

Sodium bicarbonate was blended with partial quantity (1.16%) of polyvinylpyrrolidone and granulated with a paste of the remaining polyvinylpyrrolidone in water. The wet mass was dried, milled and sifted through a 355 μm mesh (British Standard Sieve (BSS) No. 44). The sodium bicarbonate granules were blended with ofloxacin, sodium alginate, xanthan gum, carboxyvinyl polymer, cross-linked polyvinylpyrrolidone, talc and magnesium stearate and processed as described in Example 1.

The tablets were characterized for drug release in 0.1N hydrochloric acid media as described in Example 1 and the dissolution results are recorded in Table 5.

TABLE 5

| TIME (HRS) | PERCENT OFLOXACIN RELEASED |
|---|---|
| 1 | 29.1 |
| 2 | 35.2 |
| 4 | 63.1 |
| 6 | 85.7 |

It was observed that the tablets remained floating on the surface of dessolution medium till substantially all the drug was released therefrom, when they were tested without using sinkers.

### EXAMPLE 3

This example illustrates the controlled release tablets of ofloxacin wherein higher concentrations of xanthan gum were used to regulate the release progile. The pharmaceutical composition is given in Table 6.

TABLE 6

| INGREDIENTS | % W/W |
|---|---|
| Ofloxacin | 65.70 |
| Sodium alginate | 0.66 |
| Xanthan gum | 3.93 |
| Carboxyvinyl polymer (Carbopol 971P) | 2.87 |
| Cross-linked polyvinylpyrrolidone | 12.33 |
| Sodium bicarbonate | 13.11 |
| Colloidal silicon dioxide (Aerosil 200) | 0.25 |
| Magnesium stearate | 1.15 |

The tablets were prepared as described in Example 1. The tablets were characterized for drug release as disclosed in Example 1 and the dissolution results are tabulated in Table 7.

TABLE 7

| TIME (HRS) | PERCENT OFLOXACIN RELEASED |
|---|---|
| 1 | 27.3 |
| 2 | 39.8 |
| 4 | 65.4 |
| 6 | 85.5 |
| 8 | 105.8 |

### EXAMPLE 4

This example illustrates the controlled release tablets of ofloxacin wherein lower concentrations of sodium bicarbonate were used to regulate the release profile. The pharmaceutical composition is given in Table 8.

TABLE 8

| INGREDIENTS | % W/W |
|---|---|
| Ofloxacin | 72.07 |
| Sodium alginate | 0.72 |
| Xanthan gum | 2.16 |
| Carboxyvinyl polymer (Carbopol 971P) | 2.70 |
| Hydroxypropyl Methylcellulose | 0.45 |
| Cross-linked polyvinylpyrrolidone | 13.60 |
| Sodium bicarbonate | 7.21 |
| Colloidal silicon dioxide (Aerosil 200) | 0.09 |
| Magnesium stearate | 0.99 |

Sodium bicarbonate was blended with hydroxypropyl methylcellulose and granulated with purified water. The wet mass was dried, milled and sifted through a 355 μm mesh (British Standard Sieve (BSS) No.44). The sodium bicarbonate —HPMC granules were blended with ofloxacin, sodium alginate, xanthan gum, carboxyvinyl polymer, cross-linked polyvinylpyrrolidone, colloidal silicon dioxide and magnesium stearate and processed as described in Example 1.

The tablets were characterized for drug release in 0.1N hydrochloric acid as described in Example 1 and the dissolution results are recorded in Table 9.

TABLE 9

| TIME (HRS) | PERCENT OFLOXACIN RELEASED |
|---|---|
| 1 | 28 |
| 2 | 38 |
| 4 | 64 |
| 6 | 78 |
| 8 | 91 |
| 10 | 100 |

### EXAMPLE 5

This example illustrates the controlled release tablets of ofloxacin wherein the cellulosic derivative forms the integral part of the polymeric matrix. The pharmaceutical composition is given in Table 10.

TABLE 10

| INGREDIENTS | % W/W |
|---|---|
| Ofloxacin | 70.59 |
| Sodium alginate | 0.71 |
| Xanthan gum | 2.12 |
| Carboxyvinyl polymer (Carbopol 971P) | 2.65 |
| Hydroxypropyl Methylcellulose | 0.59 |
| Cross-linked polyvinylpyrrolidone | 13.47 |
| Sodium bicarbonate | 8.82 |
| Colloidal Silicon Dioxide | 0.118 |
| Magnesium stearate | 0.94 |

The tablets were prepared as described in Example 4. The tablets were characterized for drug release as disclosed in Example 1 and the dissolution results are tabulated in Table 11.

TABLE 11

| TIME (HRS) | PERCENT OFLOXACIN RELEASED |
|---|---|
| 1 | 34 |
| 2 | 52 |
| 4 | 62 |
| 6 | 75 |
| 8 | 91 |
| 10 | 101 |

### EXAMPLE 6

This examples illustrates the controlled release tablets of ofloxacin wherein lactose is used as a diluent. The pharmaceutical composition is given in Table 12.

TABLE 12

| INGREDIENTS | % W/W |
|---|---|
| Ofloxacin | 52.63 |
| Sodium alginate | 0.66 |
| Xanthan gum | 1.97 |
| Carboxyvinyl polymer (Carbopol 971P) | 3.95 |
| Hydroxypropyl Methylcellulose | 0.79 |
| Lactose monohydrate | 13.16 |
| Cross-linked polyvinylpyrrolidone | 12.76 |
| Sodium bicarbonate | 13.16 |
| Colloidal silicon dioxide (Aerosil 200) | 0.07 |
| Magnesium stearate | 0.86 |

The tablets were prepared as described in Example 4. The tablets were evaluated for release profile as disclosed in Example 1 and the dissolution are recorded in Table 13.

TABLE 13

| TIME (HRS) | PERCENT OFLOXACIN RELEASED |
|---|---|
| 1 | 31 |
| 2 | 39 |
| 4 | 57 |
| 6 | 73 |
| 8 | 87 |
| 10 | 99 |

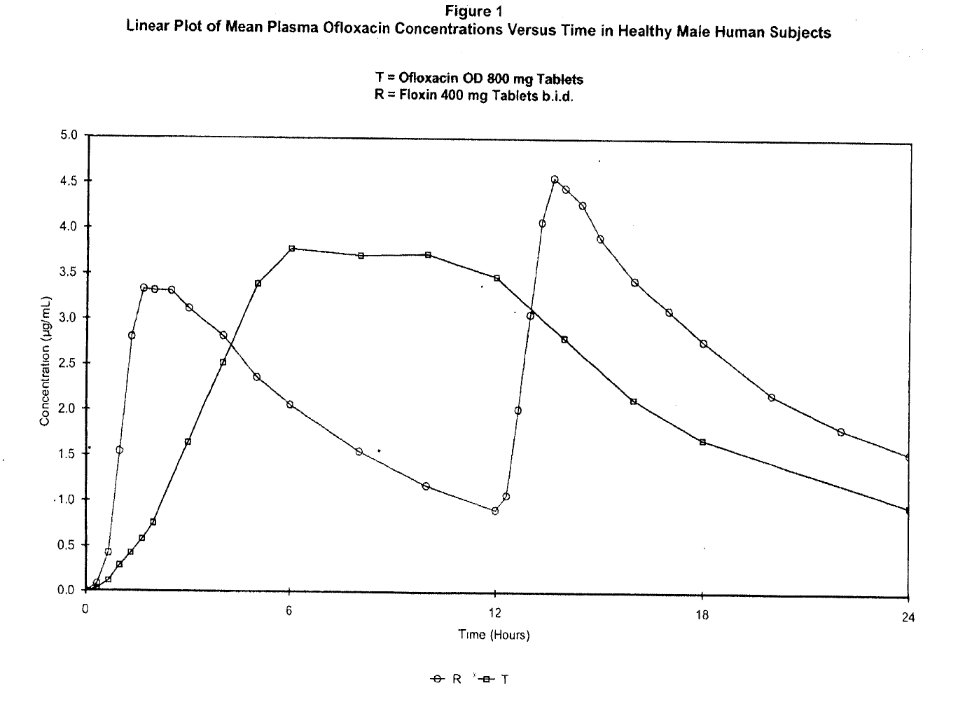

The drug release was evaluated in vivo in a randomized, two period, balanced crossover bioavailability study. The study was conducted in 24 healthy adult human subjects between 18-45 years of age where a single dose of ofloxacin OD tablets (800 mg) was administered 20 minutes after a high fat breakfast. These were compared with ofloxacin immediate release tablets (Floxin™ 400 mg, Ortho-McNeil Pharmaceutical) which were administered as a b.i.d. regimen. The first oral dose was given within 20 minutes of a high-fat breakfast and the second dose was given 12 hours later after a high-fat meal (dinner). The results of the study are shown in FIG. 1. It shows the blood profile of 800 mg ofloxacin once-a-day tablets.

The OD formulation in accordance with this invention gave a serum concentration time profile desirable for once-a-day dosage form, in that the peak serum concentration (Cmax) was comparable to that for the immediate release drug indicating a similar rate of absorption of ofloxacin. The total bioavailability of ofloxacin measured as area under the curve [AUC_{(0-∞)}] was also comparable to that of immediate release tablets given twice daily indicating that all the drug was released from the formulation and absorbed during its transit through gastrointestinal tract.

The AUC above minimum inhibitory concentration (MIC) at a levels of 1 μg/ml for ofloxacin OD also indicated comparable therapeutic efficacy to the immediate release dosage form. These results are recorded in Table 14.

TABLE 14

| | Cmax | AUC_{(0-∞)} | AUC > MIC (1 μg/ml) |
|---|---|---|---|
| Study | (μg/ml) | (μg.h/ml) | (μg.h/ml) |
| Ofloxacin 800 mg | 5.03 | 63.10 | 29.92 |
| OD | | | |
| Ofloxacin 400 mg | 4.19 | 70.36 | 31.51 |
| BID (Floxin) | | | |

Further, the extent of absorption for the test product was comparable to that for reference product as indicated by the ratio to test to reference (T/R ratio). The once-a-day tablet formulation had bio-availability of 98.19%. Thus, the therapeutic efficacy of the once-a-day dosage form as disclosed in this invention was comparable to the marketed immediate release dosage form of ofloxacin (Floxin™) given in a twice a day regimen.

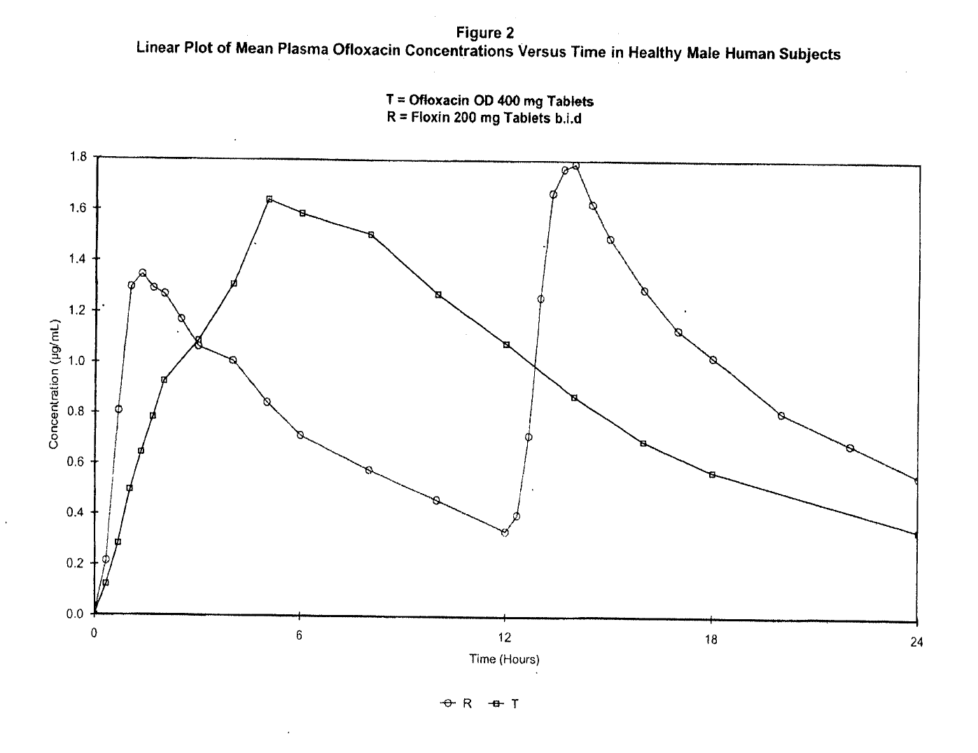

Similarly, the pharmacokintetic and pharmacodynamic parameters of once-a-day formulation (400 mg tablet) were studied in a randomized, two-period, balanced crossover bioavailability study conducted in 18 healthy, adult human subjects, between 18-45 years of age. A single dose of 400 mg of ofloxacin OD tablets was administered 20 minutes after breakfast which was compared with immediate release tablets (Floxin ™ 200 mg, Ortho McNeil Pharmaceuticals) given as a b.i.d. regimen. The first oral dose was given within 20 minutes of the breakfast and the second dose was given 12 hours later after a meal (dinner). The results of the study are recorded in FIG. 2 which shows plasma concentration over time of the dosage forms.

The OD formulations showed comparable values for Cmax, AUC and AUC above MIC as shown in Table 15.

TABLE 15

| | Cmax | AUC_{(0-∞)} | AUC > MIC (1 μg/ml) |
|---|---|---|---|
| Study | (μg/ml) | (μg.h/ml) | (μg.h/ml) |
| Ofloxacin 400 mg | 1.86 | 24.80 | 3.45 |
| OD | | | |
| Ofloxacin 200 mg | 1.56 | 25.38 | 2.57 |
| BID (Floxin) | | | |

Also, the extent of absorption of the test product was comparable to that for reference product as indicated by the T/R ratio and the formulation of the present invention had bioavailbility of 103.20%. As evident, the pharmacodynamic and pharmacokinetic parameters, which are important measures of therapeutic efficacy of the once-a-day formulation, were comparable to the marketed immediate release dosage form.

While this invention has been described with an emphasis upon preferred embodiments, it will be obvious to those of ordinary skill in the art that variations in the preferred methods of the present invention may be used and that it is intended that the invention may be practiced otherwise than as specifically described herein. Accordingly, this invention includes all modifications encompassed within the spirit and scope of the invention as defined by the following claims.

We claim:

1. A pharmaceutical composition for oral administration in humans for the controlled release of a drug or its pharmaceutically acceptable salts comprising a pharmaceutically effective amount of the drug in combination with a polymeric matrix comprising:
a carboxyvinyl polymer, said carboxyvinyl polymer forming at least 30% by weight of the total polymeric content, and
an alkaline compound.

2. The composition of claim 1 wherein the drug comprises at least one active compound selected from the therapeutic category of antiulcer, analgesic, antihypertensive, antibiotic, antipsychotic, anticancer, antimuscarinic, diuretic, antimigraine, antiviral, anti-inflammatory, sedatives, antidiabetic, antidepressant, antihistaminic, antiparasitic, antiepileptic, lipid lowering drugs, and mixtures thereof.

3. The composition of claim 1 wherein the drug is ofloxacin or its pharmaceutically acceptable salts.

4. The composition of claim 1 wherein the drug or its pharmaceutically acceptable salts form about 30% to about 90% by weight of said composition.

5. The composition of claim 1 wherein the polymeric matrix further comprises a hydrophilic polymer.

6. The composition of claim 5 wherein the hydrophilic polymer is selected from the group consisting of a cellulose ether, acrylic polymer, natural gum, and mixtures thereof.

7. The composition of claim 6 wherein the cellulose ether is selected from the group consisting of hydroxypropyl cellulose, hydroxypropyl methylcellulose, hydroxyethyl cellulose, methylcellulose, hydroxyethyl methylcellulose, hydroxypropyl, ethylcellulose, carboxymethyl cellulose, sodium carboxymethyl cellulose, hydroxycellulose, and mixtures thereof.

8. The composition of claim 6 wherein the acrylic polymer is selected from the group consisting of methacrylates, polyacrylates copolymers, and mixtures thereof.

9. The composition of claim 6 wherein the natural gum is selected from the group consisting of xanthan gum, karaya gum, locust bean gum, guar gum, gelan gum, gum arabic, tragacanth, carrageenan, pectin, agar, alginic acid, sodium alginate, and mixtures thereof.

10. The composition of claim 1 wherein the total polymeric content is about 2% to about 25% by weight of said composition.

11. The composition of claim 1 wherein the total polymeric content is about 5% to about 15% by weight of said composition.

12. The composition of claim 1 wherein the alkaline compound is an inorganic basic salt or an organic basic salt.

13. The composition of claim 12 wherein the alkaline compound is a gas generating agent.

14. The composition of claim 13 wherein the gas generating agent is a sulfite, a carbonate or a bicarbonate salt.

15. The composition of claim 14 wherein the gas generating agent is selected from the group consisting of sodium bicarbonate, potassium bicarbonate, sodium glycine bicarbonate, calcium carbonate, ammonium bicarbonate, sodium sulfite, sodium bisulfite and sodium metabisulfite.

16. The composition of claim 13 wherein the gas generating agent is a gas couple comprising a gas generating salt and an edible organic acid or a salt of an edible organic acid.

17. The composition of claim 16 wherein the edible organic acid is selected from the group consisting of citric acid, ascorbic acid, tartaric acid, succinic acid, fumaric acid, malic acid, maleic acid and their salts, glycine, sarcosine, alanine, taurine and glutamic acid.

18. The composition of claim 1 wherein the alkaline compound forms about 5% to about 50% by weight of said composition.

19. The composition of claim 1 wherein the alkaline compound forms about 10% to about 30% by weight of said composition.

20. The composition of claim 1 wherein the polymeric matrix further comprises pharmaceutically acceptable auxiliary components including swelling agents.

21. The composition of claim 20 wherein the swelling agent comprises a superdisintegrant.

22. The composition of claim 21 wherein the superdisintegrant is selected from the group consisting of cross-linked polyvinylpyrrolidone, cross-linked sodium carboxymethyl cellulose, sodium starch glycolate, and mixtures thereof.

23. The composition of claim 20 wherein the swelling agent forms about 5% to about 30% by weight of said composition.

24. The composition of claim 23 wherein the swelling agent forms about 10% to about 20% by weight of said composition.

25. The composition of claim 20 wherein the dosage form further comprises diluents, binder, glidant, anti-adherent, lubricant or mixtures thereof.

26. A pharmaceutical composition for oral administration in humans for the controlled release of ofloxacin which releases more than 40% of ofloxacin in less than 4 hours and releases more than 60% of ofloxacin in less than 8 hours.

27. A pharmaceutical composition for oral administration in humans for the controlled release of ofloxacin which releases more than 40% of ofloxacin within about 2-4 hours and releases more than 60% of ofloxacin within about 4-8 hours.

28. The composition of claims 24 or 25 wherein the dosage form is formed into a physical form selected from the group consisting of pellets, beads, granules, tablets and capsules.

29. The composition according to claim 28 wherein the capsule shell comprises a substance selected from the group consisting of gelatin, hydroxypropyl methylcellulose, or starch.

30. The composition of claim 28 wherein the tablet dosage form further comprises a coating with a fast dissolving film of a water soluble polymer.

31. A pharmaceutical composition for oral administration in humans for a once-a-day delivery system of a drug comprising:
a pharmaceutically effective amount of the drug or its pharmaceutically acceptable salts;
a polymeric matrix wherein at least one polymer is carboxyvinyl polymer, the carboxyvinyl polymer forming at least 30% by weight of the total polymeric content;
cellulose ether; and
an alkaline compound.

32. The composition of claim 31 wherein the drug comprises at least one active compound selected from the therapeutic category of antiulcer, analgesic, antihypertensive, antibiotic, antipsychotic, anticancer, antimuscarinic, diuretic, antimigraine, antiviral, anti-inflammatory, sedatives, antidiabetic, antidepressant, antihistaminic, antiparasitic, antiepileptic, lipid lowering drugs, and mixtures thereof.

33. The composition of claim 31 wherein the drug is ofloxacin or its pharmaceutically acceptable salts.

34. The composition of claim 31 wherein drug comprises about 30% to about 90% by weight of said composition.

35. The composition of claim 31 wherein the cellulose ether is selected from the group consisting of hydroxypropyl cellulose, hydroxypropyl methylcellulose, hydroxyethyl cellulose, methylcellulose, hydroxyethyl methylcellulose, hydroxypropyl ethylcellulose, carboxymethyl cellulose, sodium carboxymethyl cellulose, hydroxycellulose, and mixtures thereof.

36. The composition of claim 31 wherein the polymeric matrix further comprises a hydrophilic polymer.

37. The composition according to claim 36 wherein the hydrophilic polymer comprises an acrylic polymer, natural gum, and mixtures thereof.

38. The composition according to claim 37 wherein the acrylic polymer is selected from the group consisting of methacrylates, polyacrylates copolymers, and mixtures thereof.

39. The composition according to claim 37 wherein the natural gum is selected from the group consisting of xanthan gum, karaya gum, locust bean gum, guar gum, gelan gum, gum arabic, tragacanth, carrageenan, pectin, agar, alginic acid, sodium alginate, and mixtures thereof.

40. The composition according to claim 31 wherein the total polymeric content is about 2% to about 25% by weight of said composition.

41. The composition according to claim 31 wherein the total polymeric content is about 5% to about 15% by weight of said composition.

42. The composition according to claim 31 wherein the alkaline compound is an inorganic basic salt or an organic basic salt.

43. The composition according to claim 42 wherein the alkaline compound is more preferably a gas generating agent.

44. The composition according to claim 43 wherein the gas generating agent is a sulfite, a carbonate or a bicarbonate salt.

45. The composition according to claim 44 wherein the gas generating agent is selected from the group consisting of sodium bicarbonate, potassium bicarbonate, sodium glycine bicarbonate, calcium carbonate, ammonium bicarbonate, sodium sulfite, sodium bisulfite and sodium metabisulfite.

46. The composition according to claim 43 wherein the gas generating agent is a gas couple comprising a gas generating salt and an edible organic acid or a salt of an edible organic acid.

47. The composition according to claim 46 wherein the edible organic acid is selected from the group consisting of citric acid, ascorbic acid, tartaric acid, succinic acid, fumaric acid, malic acid, maleic acid or their salts, glycine, sarcosine, alanine, taurine and glutamic acid.

48. The composition according to claim 31 wherein the alkaline compound comprises about 5% to about 50% by weight of said composition.

49. The composition according to claim 31 wherein the alkaline compound comprises about 10% to about 30% by weight of said composition.

50. The composition according to claim 31 wherein the polymeric matrix further comprises pharmaceutically acceptable auxiliary components, which comprise swelling agents.

51. The composition according to claim 50 wherein the swelling agent comprises a superdisintegrant.

52. The composition to claim 51 wherein the superdisintegrant is selected from the group consisting of cross-linked polyvinylpyrrolidone, cross-linked sodium carboxymethyl cellulose, sodium starch glycolate, and mixtures thereof.

53. The composition according to claim 50 wherein the swelling agent comprises about 5% to about 30% by weight of said composition.

54. The composition according to claim 53 wherein the swelling agent comprises about 10% to about 20% by weight of said composition.

55. The composition according to claim 50 wherein the dosage form further comprises diluents, binder, glidant, anti-adherent, lubricant or mixtures thereof.

56. A pharmaceutical composition for oral administration in humans for the once-a-day delivery of ofloxacin which releases more than 40% of ofloxacin in less than 4 hours, releases more than 60% of ofloxacin in less than 8 hours and substantially all ofloxacin is released in about 10 hours.

57. A pharmaceutical composition for oral administration in humans for the once-a-day delivery of ofloxacin which releases more than 40% of ofloxacin within about 2-4 hours, releases more than 60% of ofloxacin within about 4-8 hours and substantially all ofloxacin is released within about 8-10 hours.

58. A once-a-day dosage form of ofloxacin which, when orally administered in humans under fed conditions, provides mean peak serum concentration, area under the serum concentration-time curve above minimum inhibiting concentrations and durations above minimum inhibitory serum concentrations, of not less than 70% when compared with respect to divided dose of an equivalent amount of conventional immediate release ofloxacin dosage form.

59. A pharmaceutical composition according to claim 31 wherein the dosage form is formed into a physical form selected from the group consisting of pellets, beads, granules, tablets and capsules.

60. The composition according to claim 59 wherein the capsule shell is made of gelatin, hydroxypropyl methylcellulose or starch.

61. The composition according to claim 59 wherein the tablet further comprises coating with a fast dissolving film of a water soluble polymer.
